(19) United States
(12) Patent Application Publication
(10) Pub. No.: US 2025/0014046 A1
(43) Pub. Date: Jan. 9, 2025

(54) SYSTEM, METHOD AND PROGRAM PRODUCT FOR PROVIDING ABSORBENT PRODUCT SUPPORT INFORMATION

(71) Applicant: First Quality Enterprises, Inc., Great Neck, NY (US)

(72) Inventors: Michele Anne Mongillo, Bradford, PA (US); Kimberly Crews Owen, Evington, VA (US)

(21) Appl. No.: 18/762,041

(22) Filed: Jul. 2, 2024

Related U.S. Application Data
(60) Provisional application No. 63/525,073, filed on Jul. 5, 2023.

(51) Int. Cl. G06Q 30/015; H04L 51/046

(52) U.S. Cl. CPC G06Q 30/015; H04L 51/046

(57) ABSTRACT

A computer system for providing disposable absorbent product selection and user support including: a compilation of product descriptions, sizing, instructions for use, and training materials; at least one toll free phone number for calling to receive assistance; at least one website chat feature for accessing assistance; and at least two clinicians and/or licensed nurses for receiving and responding to questions.

### RELATED APPLICATIONS

This application claims priority to and the benefit of U.S. Provisional Application No. 63/525,073, filed Jul. 5, 2023 and entitled SYSTEM, METHOD AND PROGRAM PRODUCT FOR PROVIDING ABSORBENT PRODUCT SUPPORT INFORMATION the contents of which are incorporated herein by reference in their entirety.

### FIELD OF THE INVENTION

The present invention generally relates to a systems, methods and program products for providing support to product distributors and consumers using absorbent products.

### BACKGROUND

Disposable absorbent products including disposable diapers, training pants, briefs, bladder control pads and the like have been made and sold for many years. The products come in many sizes and are designed with different absorbencies, fastening mechanisms and other features for different purposes. Technology and materials have evolved such that these disposable absorbent products have become a growing market.

Disposable absorbent products may be manufactured by a company that sells the product directly to consumers and/or indirectly to consumers through medical equipment and supplies distributors. Due to the continuous improvement in disposable absorbent products, there is a need for manufacturers to be able to provide useful information on the products, such as appropriate selection, sizing and the like. Traditionally, this information is provided on product packages. It can be overwhelming to compare product packages to try to determine which is the best product for yourself or a loved one.

First Quality Products (Great Neck, NY, USA) launched an eNurse® system for providing support to medical equipment and supplies distributors and consumers to assist in purchasing decisions and how to use disposable absorbent products. In accordance with the system, nurses visit healthcare sites where the products are sold and utilized and offer support and training on selecting and using disposable absorbent products. A toll-free phone number is provided that connects the user to a licensed nurse trained to answer questions relating to disposable incontinence products and their use. The toll-free number is available 7 days a week and 24 hours a day. The number is used to provide support relating to PREVAIL® brand disposable incontinence products. Consumers at home, case managers, customer service representatives, nursing home staff and the like have access to the toll-free number and support.

A technological problem exists with conventional absorbent product support systems, such as the eNurse system, in that customers are not able to obtain information and support on a variety of different available brands and types of absorbent products through a single information hub. As the market is growing and many products are not included in the existing system, there is a need for an improved disposable absorbent product support system.

### SUMMARY OF THE INVENTION

In exemplary embodiments, the present invention provides a technological solution to the above-described technological problem by providing a central computerized information hub through which consumers, case managers, customer service representatives, nursing home staff and the like may access information about and obtain support for absorbent products available from a variety of suppliers and distributors. In exemplary embodiments, the system is computer automated so that users are directed to appropriate information sources depending on the specific request for information and support. As opposed to needing to obtain information from a variety of sources, the central computer hub in accordance with exemplary embodiments of the present invention maintains a central database of information pertaining to a variety of products, brands, suppliers, and distributors, and thus the computer system is able to operate more efficiently in obtaining requested information.

The present invention provides a system for providing disposable absorbent product selection and use support including a compilation of product descriptions, sizing, instructions for use, and training materials; at least one toll free phone number for calling to receive assistance; at least one website chat feature for accessing assistance and at least 2 clinicians and/or nurses for receiving and responding to questions.

According to an exemplary embodiment of the present invention, a computer system for providing disposable absorbent product selection and use support comprises: one or more processors; and non-transitory computer-readable memory operatively connected to the one or more processors and having stored thereon machine-readable instructions that, when executed by the one or more processors, cause the one or more processors to perform a method comprising: (A) receiving, at one or more computers, from a customer, a request for information related to at least one of a plurality of disposable absorbent products; (B) providing, by the one or more computers, a response to the request for information, the step of providing comprising: (1) accessing, by one or more computers, one or more databases containing information pertaining to a plurality of disposable absorbent products, including one or more of the following: (i) pictures of the plurality of disposable products; (ii) description of components corresponding to each disposable absorbent product; (iii) instructions for use corresponding to each disposable absorbent product; (iv) training material corresponding to each disposable absorbent product; and (v) contact information associated with each disposable absorbent product; (2) retrieving, by the one or more computers, response data from the one or more databases responsive to the request for information; (3) providing, by the one or more computers, the response data to the customer; (4) determining, by the one or more computers, whether the response data is satisfactory; (5) if the response data is determined to be not satisfactory, accessing, by the one or more computers, the one or more databases to determine an appropriate clinician or other representative to provide the requested information; (6) if the response data is determined to be satisfactory, determining, by the one or more computers, that the request for information is complete.

In an exemplary embodiment, the steps (A) and/or (B) of receiving and/or providing comprises providing, by the one or more computers, a webpage or a software application.

In an exemplary embodiment, the steps (A) and/or (B) of receiving and/or providing comprises providing, by the one or more computers, a website chat feature.

In an exemplary embodiment, the website chat feature is a chatbot. The chatbot is a Machine Learning based software system that leverages commercially available Natural Language Processing and Natural Language Understanding Artificial Intelligence technology to answer incontinence related questions that has been trained based on Incontinence body of knowledge.

### BRIEF DESCRIPTION OF THE DRAWINGS

FIG. 1 is a block diagram showing a general environment in which a disposable absorbent product support computer system operates in accordance with an exemplary embodiment of the present invention; and

FIG. 2 is a flowchart showing operation of a disposable absorbent product support computer system according to an exemplary embodiment of the present invention.

### DETAILED DESCRIPTION OF PREFERRED EMBODIMENTS

The system of the present invention provides useful disposable absorbent product information to consumers. In exemplary embodiments, the information is provided by clinicians and/or nurses from the compilation of information. The compilation includes pictures of products with descriptions of the components in the products. The product absorbency and recommended use is also provided. A description of how the product is applied and worn may include sketches with step-by-step procedures for application and use. Where appropriate, videos demonstrating proper application are provided. The compilation of information also includes cautions for safe use and disposal instructions. The compilation of information may be stored on a hard drive, stand-alone server or cloud-based server. The user may access the system through their home computer, tablet, or mobile phone. The system may apply broadly to disposable absorbent products and may be particularly useful for disposable absorbent products used with incontinence. The system may also be useful for baby products such as disposable baby diapers, training pants, wipes and the like. A list of typical questions the support system provides answers for includes, but is not limited to:
What is incontinence?
Why am I having incontinence issues?
How can I prevent incontinence?
What are different treatment options?
What products do you recommend?
Which product should I use?
Which product is more absorbent, discrete, sensitive?
What is the right size for me?
What are your products made up of?
Where can I buy incontinence products?
What's the right way to wear your products?
How long should I wear your products?
Insurance questions.
How can I obtain product samples?

The clinicians and/or licensed nurses may be bi-lingual or multi-lingual and may field calls or chats directly from consumers who find a toll-free number on absorbent product packaging or on an internet website where the products are sold. The calls or chats may come from the manufacturer's branded products customers. The system may be set up such that the licensed nurses and/or clinicians—may field calls or chats from customers of store brands that their company manufactures products for. The system is set up such that the clinician and/or licensed nurse knows what brand of product the caller is inquiring about. This may be accomplished through having dedicated call lines for different brands or indicators on a dashboard the nurse or clinician is using. The dashboard may be associated with software that also houses the compilation information. When the software is not running, the clinician or nurse may identify themselves generically rather than as brand X support.

Given the potential volume of calls, the system may include multiple toll-free lines, for example from 2 to 20 or 5 to 15 or 12 lines. The lines are staffed by nurses and/or clinicians working different time shifts such that support may be provided 24 hours a day, 7 days a week. The system may be set up to enable out of country calls and chats, for example, consumers in Canada or Mexico may access the system to obtain useful information. The system may include software for virtual meetings such as Microsoft TEAMS software, Zoom Video Communications ZOOM software and the like.

In order to facilitate more sharing of disposable absorbent product selection and use information, licensed nurses and/or clinicians may conduct virtual video visits with nursing home customers to provide education/consultation. To enhance the system, chatbots may be used to provide additional support and make the system more efficient. A chatbot (also known as a chatterbot) is a software application that is used to conduct an online chat conversation via typing or typing-to-speech, instead of providing direct contact with a live human. The chatbots utilize information from the compilation utilizing commercially available software loaded with the information. This also limits live repetitive interactions and provides efficiency.

In exemplary embodiments of the present invention, the chatbot is powered by a machine learning model using commercially available Natural Language Understanding and Processing (NLU & NLP) as well as Question and Answering models. The machine learning model may be trained with a proprietary Knowledge Base about Incontinence as well as operating processes created and operated by First Quality (Free Samples offered via Prevail) and First Quality's distribution network (where First Quality product can be purchased).

With advances in artificial intelligence, it is possible to provide additional support through the use of a digital human or bot. These may also provide support in many languages to expand support to customers in other countries. The system may facilitate providing support to international retail for big box stores. When the digital human or bot is not able to answer a customer question, the system may forward the call or chat to a live nurse or clinician.

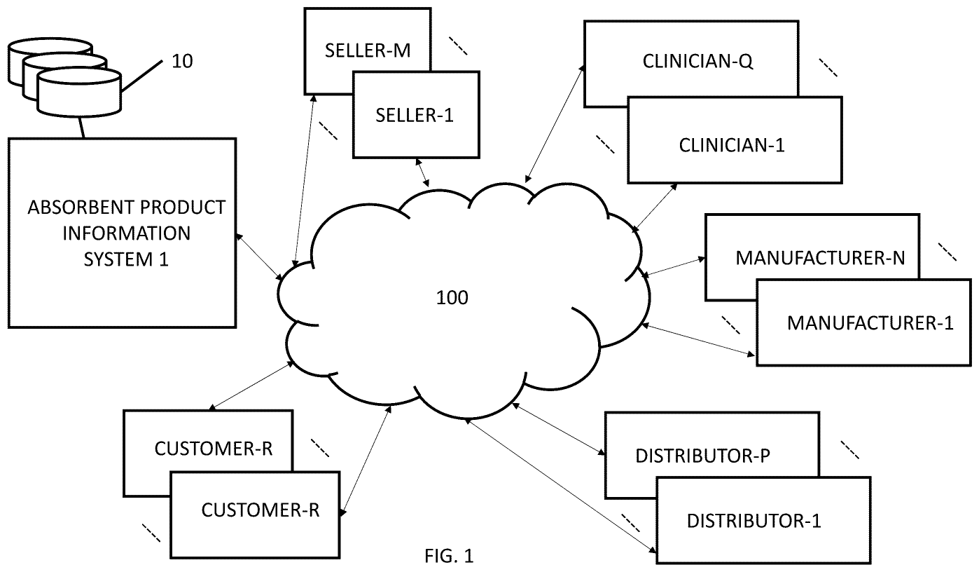

FIG. 1 is a flowchart demonstrating how the system of the present invention provides support to consumers needing information. The consumer may call a toll free number shown on disposable absorbent product packaging or in a website and talk to a nurse or clinician. Alternatively, the consumer may select a chat function in a website and communicate with human support, a chatbot or digital human. The nurse or clinician, or chatbot or digital human receives questions from the customer and obtains relevant information from the compilation of useful information, then responds to the customer with the information. In the event that the chatbot or digital human is not able to answer a question, the customer is forwarded to a nurse or clinician.

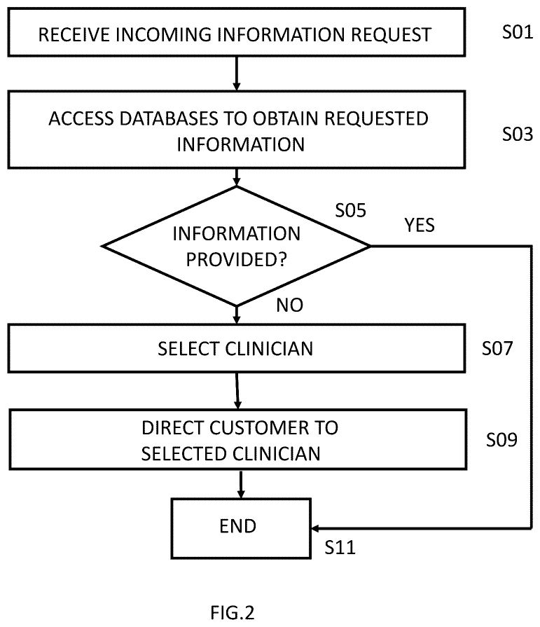

FIG. 2 is a block diagram showing the interaction between an absorbent product support computer system 1, one or more absorbent product seller computer systems (seller-1 . . . seller-M), one or more absorbent product manufacturer computer systems (manufacturer-1 . . . manufacturer-N), one or more absorbent product distributor computer systems (distributor-1 . . . distributor-P), one or more clinician computer systems (clinician-1 . . . clinician-Q) and one or more customer computer systems (customer-1 . . . customer-R) according to an exemplary embodiment of the present invention. The absorbent product support computer system 1 is configured to provide disposable absorbent product information to the customers, including, for example, disposable absorbent product selection and user support including a compilation of product descriptions, sizing, instructions for use, and training materials, to name a few. In this regard, customers may access the absorbent product support computer system 1 and obtain information from a variety of sources, including, for example, absorbent product sellers, absorbent product manufacturers, absorbent product distributors, and clinicians (e.g., nurses or other clinicians trained to answer questions relating to disposable incontinence products and their use).

All computers, computer systems, and/or user devices described herein may comprise one or more processors and non-transitory computer-readable memory (e.g., local and/or remote memory) having stored thereon computer-readable instructions to perform the processes described herein with respect to each device and/or computer system. In embodiments, various processing may be performed by particularly programmed software agents or software modules. Each device and/or computer system may store data in its respective memory, which may be organized in one or more databases. Each device and/or computer system may also have one or more input devices (e.g., touchscreen, pointer device, mouse, keyboard, microphone, camera, video camera, to name a few) and/or one or more output devices (e.g., display screens, projectors, speakers). In embodiments, computer systems may comprise one or more servers or server farms, which may not have physical input or output devices directly connected thereto or embedded therein.

Each device and/or computer system may also include one or more communication portals. Accordingly, the devices and/or computer systems may be operatively connected directly, e.g., via wired or wireless communications, and/or indirectly, e.g., via a data network 100, such as the Internet, a telephone network, a mobile broadband network (e.g., a cellular data network), a mesh network, a local area network (LAN) (including a wireless local area network, e.g., a Wi-Fi network), a wide area network (WAN), a metropolitan area network (MAN), and/or a global area network (GAN), to name a few. Data networks may be provided via wired and/or wireless connections. Data networks may be public or private. Accordingly, data networks may be open or closed, such as requiring authorized access, specific communication connections, or specialized hardware and/or software. In embodiments, any combination of communications channels may be utilized.

The respective communications portals of each computer system and/or user device may handle, process, support, and/or perform wired and/or wireless communications, such as transmitting and/or receiving data (e.g., data packets). In embodiments, transmission described with respect to a single data packet may comprise a plurality of data packets. Data packets may be discrete electronic units of data. In other embodiments, transmissions may comprise non-discrete signals, such as data streams. Transmissions described with respect to data packets may also comprise data transmissions via other communications mechanisms known in the art, such as data streams. Communications portals can comprise hardware (e.g., hardware for wired and/or wireless connections, such as communications chipsets, communications interfaces, and/or communications antennas, to name a few) and/or software.

Wired connections may be adapted for use with cable, plain old telephone service (POTS) (telephone), fiber (such as Hybrid Fiber Coaxial), xDSL, to name a few, and wired connections may use coaxial cable, fiber, copper wire (such as twisted pair copper wire), and/or combinations thereof, to name a few. Wired connections may be provided through telephone ports, Ethernet ports, USB ports, and/or other data ports, such as Apple 30-pin connector ports or Apple Lightning connector ports, to name a few. Wireless connections may include cellular or cellular data connections and protocols (e.g., digital cellular, PCS, CDPD, GPRS, EDGE, CDMA2000, 1.times.RTT, Ev-DO, HSPA, UMTS, 3G, 4G, 5G, and/or LTE, to name a few), Bluetooth, Bluetooth Low Energy, Wi-Fi, radio, satellite, infrared connections, ZigBee communication protocols, to name a few. Communications interface hardware and/or software, which may be used to communicate over wired and/or wireless connections, may comprise Ethernet interfaces (e.g., supporting a TCP/IP stack), X.25 interfaces, TI interfaces, and/or antennas, to name a few. In exemplary embodiments, communications may be encrypted by encryption techniques, such as, for example, symmetrical encryption or public key encryption.

The absorbent product information computer system 1 may include a computer system having a non-transitory computer-readable memory, which may store data, e.g., in one or more databases or data stores 10. Accordingly, the computer system 1 can store various types of absorbent product information data and other variables, as described herein in connection with embodiments of the present invention. According to an exemplary embodiment, the system 1 stores data pertaining to information related to a variety of different sellers, distributors and manufacturers of absorbent products and the types of absorbent products made available by those sellers, distributors and manufacturers.

The absorbent product support computer system 1 may be configured to provide automated responses to incoming calls, emails, texts or other electronic communications to provide requested information to customers and/or direct customers to clinicians who can answer questions and/or otherwise provide guidance on absorbent products and their proper use. In an exemplary embodiment, the absorbent product support computer system 1 may perform the process steps as shown in FIG. 2. Specifically, at step S01, the system 1 may receive an incoming request for information regarding an absorbent article from a customer in the form of, for example, a phone call, a text or an email. In this regard, the customer may access a webpage at a website or dedicated application (e.g., a mobile phone software application or “app”) through which a request may be entered or selected from a list of available requests for information. The webpage may provide categories and sub-categories that the customer may browse through to focus the request on a particular manufacturer, vendor, distributor and/or product. After receiving the request, the system 1 may at step S03 access one or more databases to obtain information related to the request using, for example, a lookup operation. At step S05, if the system 1 determines that the requested information is not available and/or the request response is not satisfactory to the customer, the system 1 may at step S07 again access one or more databases to determine an appropriate clinician or other representative that might be able to provide the appropriate information. In this regard, the system 1 may prompt the customer to answer whether the provided information is satisfactory. If the requested information is satisfactory, the process may end at step S11. Otherwise, at step S09, the system 1 may place the customer in contact with the selected clinician either by phone, text, email or other electronic communication. The process may then end at step S11, at which point the selected clinician or other representative may provide the requested information.

Now that embodiments of the present invention have been shown and described in detail, various modifications and improvements thereon can become readily apparent to those skilled in the art. Accordingly, the exemplary embodiments of the present invention, as set forth above, are intended to be illustrative, not limiting. The spirit and scope of the present invention is to be construed broadly.

We claim:

1. A computer system for providing disposable absorbent product selection and use support comprising:
one or more processors; and
non-transitory computer-readable memory operatively connected to the one or more processors and having stored thereon machine-readable instructions that, when executed by the one or more processors, cause the one or more processors to perform a method comprising:
(A) receiving, at one or more computers, from a customer, a request for information related to at least one of a plurality of disposable absorbent products;
(B) providing, by the one or more computers, a response to the request for information, the step of providing comprising:
(1) accessing, by one or more computers, one or more databases containing information pertaining to a plurality of disposable absorbent products, including one or more of the following:
(i) pictures of the plurality of disposable products;
(ii) description of components corresponding to each disposable absorbent product;
(iii) instructions for use corresponding to each disposable absorbent product;
(iv) training material corresponding to each disposable absorbent product; and
(v) contact information associated with each disposable absorbent product;
(2) retrieving, by the one or more computers, response data from the one or more databases responsive to the request for information;
(3) providing, by the one or more computers, the response data to the customer;
(4) determining, by the one or more computers, whether the response data is satisfactory;
(5) if the response data is determined to be not satisfactory, accessing, by the one or more computers, the one or more databases to determine an appropriate clinician or other representative to provide the requested information;
(6) if the response data is determined to be satisfactory, determining, by the one or more computers, that the request for information is complete.

2. The system of claim 1, wherein the steps (A) and/or (B) of receiving and/or providing comprises providing, by the one or more computers, a webpage or a software application.

3. The system of claim 1, wherein the steps (A) and/or (B) of receiving and/or providing comprises providing, by the one or more computers, a website chat feature.

4. The system of claim 3, wherein the website chat feature is a texting feature or a chatbot.
